(19) United States
(12) Patent Application Publication
(10) Pub. No.: US 2020/0000645 A1
(43) Pub. Date: Jan. 2, 2020

(54) METHOD FOR REDUCING BLOOD LOSS

(71) Applicant: Rhinostasis, Fruit Heights, UT (US)

(72) Inventors: Jacob Hess, Fruit Heights, UT (US); Dalton Facer, Fruit Heights, UT (US); Kortney James, Fruit Heights, UT (US)

(21) Appl. No.: 16/258,964

(22) Filed: Jan. 28, 2019

Related U.S. Application Data
(60) Provisional application No. 62/622,216, filed on Jan. 26, 2018.

(51) Int. Cl. A61F 13/20

(52) U.S. Cl. CPC A61F 13/2005; A61B 17/12104; A61F 13/2074

(57) ABSTRACT

The claimed method relates to a method for reducing blood loss. More specifically, the claimed method utilizes at least one compound deposited within and/or on a medical applicator to reduce blood loss experienced by a user. In some embodiments, the method may include depositing at least one compound onto or within a medical applicator which promotes diminished blood flow and coagulation when the applicator is used. More specifically, the medical applicator may have a compound adhered to the surface which acts as a catalyst to instigate blood clotting when the medical applicator is used for its intended purpose. In some embodiments, an antifibrinolytic may be deposited onto the medical applicator. In other embodiments, compounds causing vasoconstriction may be deposited onto the medical applicator. In yet other embodiments, a plurality of compounds may be deposited within and/or on the surface of the medical applicator.

### RELATED CASES

This application claims priority to U.S. Provisional Application No. 62/622,216, filed on Jan. 26, 2018, and titled “Method for Reducing Blood Loss,” which is hereby incorporated by reference in its entirety.

### BACKGROUND

### A. Field of the Invention

The present invention relates to a method for reducing blood loss. More specifically, the claimed method utilizes at least one compound deposited within and/or on medical applicators to reduce blood loss experienced by the user.

### B. Background

When blood vessels are injured, they release blood which carries platelets and proteins that form a clot over the injured blood vessels to repair the damage and prevent continued bleeding. Many people have blood conditions that inhibit or dramatically lessen their blood's ability to clot to prevent continuous bleeding from injured blood vessels. For example, the bleeding individual may have a clotting disorder or be taking medications that have the effect of thinning the user's blood. For this reason, even trivial blood loss such as a nosebleed or a minor wound can be very serious and require hospitalization for people who have blood clotting problems. While nasal tampons and medical bandages (e.g., gauze, compression, etc.) may be used to stifle bleeding, having blood that cannot clot or cannot rapidly clot still poses a very serious health risk. Often, individuals who have blood clotting complications require costly emergency medical treatment.

### SUMMARY

This summary of this method is not intended to describe each illustrated embodiment or every possible implementation of the method. The figures and the detailed description that follow, however, do particularly exemplify these embodiments. The method, which relates to reducing a blood loss experienced by a user, discloses a method in which at least one compound may be deposited within and/or on a medical applicator to give the applicator an additional characteristic for reducing blood loss experienced by the user.

The method may include depositing at least one compound onto or within a medical applicator which promotes diminished blood flow and/or coagulation when the applicator is used. More specifically, the medical applicator may have a compound deposited onto the surface or deposited within the applicator which acts as a catalyst to instigate blood clotting when the medical applicator is used for its intended purpose. For example, nasal tampons are commonly used to treat nosebleeds and in one embodiment, a nasal tampon having an antifibrinolytic compound (e.g., tranexamic acid) deposited onto its surface may be used to inhibit the flow of blood from a patient's nose while simultaneously promoting the formation of a blood clot.

Additional features and advantages of the method will be set forth in the description which follows, and in part will be obvious from the description, or may be learned by the practice of the invention. The features and advantages of the method may be realized and obtained by means of the instruments and combinations particularly pointed out in the appended claims. These and other features of the method will become more fully apparent from the following description and appended claims, or may be learned by the practice of the invention as set forth hereinafter.

### BRIEF DESCRIPTION OF THE DRAWINGS

In consideration of the following detailed description of various embodiments, the claimed method may be more completely understood in connection with the following drawings.

FIG. 1A is a side view of an embodiment of an elongate medical applicator.

FIG. 1B is a distal end view of an embodiment of the elongate medical applicator of FIG. 1A.

FIG. 2A is a side view of an embodiment of a medical applicator that comprises an absorptive material and a rigid member and a pull string.

FIG. 2B is a side view of the medical applicator of FIG. 2A after the absorptive material has expanded.

FIG. 2C is a side view of the medical applicator of FIG. 2A after the absorptive material has expanded.

FIG. 2D is a perspective view of the medical applicator of FIG. 2A.

FIG. 3A is a side view of an embodiment of a medical applicator vaginal tampon.

FIG. 3B is a distal end view of the medical applicator vaginal tampon.

FIG. 4A is a side view of an embodiment of a medical applicator gauze pad.

FIG. 4B is a side view of an embodiment of a medical applicator bandage roll.

FIG. 4C is an end view of the medical applicator bandage roll of FIG. 4B.

FIG. 5 is an embodiment of a medical applicator adhesive bandage.

FIG. 6A is a side view of an embodiment of an elongate medical applicator that comprises a cross-bar.

FIG. 6B is a bottom view of the elongate medical applicator of FIG. 6A.

FIG. 6C is a side view of the elongate medical applicator of FIG. 6A.

FIG. 6D is an exploded view of the medical applicator of FIG. 6A.

FIG. 7 illustrates a method of depositing compounds or pharmaceutical agents on the outer surface of within a medical applicator to reduce blood loss.

Although the method is amenable to various modifications and alternative forms, specifics thereof have been shown by way of example in the drawings and will be described in detail such that the disclosure will be thorough and complete, and will fully convey the scope of the claimed method to those skilled in the art. However, it should be understood, that the intention is not to limit the method to the particular embodiments described. On the contrary, the intention is to cover all modifications, equivalents, and alternatives falling within the spirit and scope of the method as defined by the appended claims.

### DETAILED DESCRIPTION

The components of the embodiments as generally described and illustrated in the figures herein can be arranged and designed in a wide variety of different configurations. Thus, the following more detailed description of various embodiments, as represented in the figures, is not intended to limit the scope of the present disclosure, but is merely representative of various embodiments. While various aspects of the embodiments are presented in drawings, the drawings are not necessarily drawn to scale unless specifically indicated.

The phrase “coupled to” is broad enough to refer to any suitable coupling or other form of interaction between two or more entities, including mechanical, fluidic and thermal interaction. Thus, two components may be coupled to each other even though they are not in direct contact with each other. The phrases “attached to” or “attached directly to” refer to interaction between two or more entities which are in direct contact with each other and/or are separated from each other only by a fastener of any suitable variety (e.g., mounting hardware or an adhesive).

The terms “proximal” and “distal” are opposite directional terms. For example, the distal end of a device or component is the end of the component that is furthest from the practitioner during ordinary use. The proximal end refers to the opposite end, or the end nearest the practitioner during ordinary use.

As discussed previously, many people have blood conditions that inhibit or dramatically lessen their blood's ability to clot and prevent continuous bleeding from injured blood vessels. This inability may be caused by a clotting disorder or medications that have an undesired side effect of thinning the user's blood (e.g., Warfarin, Coumadin, Xarelto, and Eliquis). Even trivial blood loss such as nosebleeds or minor injuries can be very serious and require hospitalization for people who have blood clotting problems. While nasal tampons and medical bandages (e.g., gauze, compression, etc.) may be used to stifle bleeding, having blood that cannot clot or cannot rapidly clot still poses a very serious health risk. Thus, there is a need for a method to reduce blood loss.

The claimed method relates to a method for reducing blood loss. More specifically, the claimed method utilizes at least one compound deposited within and/or on medical applicators to reduce blood loss experienced by a user. In some embodiments, the method may include depositing at least one compound onto or within a medical applicator (e.g., nasal tampons, vaginal tampons, gauze, etc.) which promotes diminished blood flow and coagulation when the applicator is used. More specifically, the medical applicator may have a compound adhered to the surface which acts as a catalyst to instigate blood clotting when the medical applicator is used for its intended purpose. In some embodiments, an antifibrinolytic may be deposited onto the medical applicator. Antifibrinolytics such as aminocaproic acid or tranexamic acid act to inhibit the body's natural breakdown of blood clots. In other embodiments, compounds causing vasoconstriction may be deposited onto the medical applicator. Compounds causing vasoconstriction such as Oxymetazoline act to contract the muscular wall of the blood vessel and thereby restrict blood flow. In yet other embodiments, a plurality of compounds may be deposited within and/or on the surface of the medical applicator.

The compounds may be deposited on and/or within the medical applicator in a variety of ways. For example, the compound may be freeze dried onto and/or within the medical applicator through a lyophilizing process, in one embodiment. In another embodiment, the compound may be deposited in the applicator by mixing the compound with a liquid to form a solution, soaking the applicator in the solution, and evaporating the liquid from the medical applicator. In yet another embodiment, the compound may be mixed with a liquid to form a solution and the medical applicator may be soaked therein prior to packaging. It should be appreciated that any means to deposit the compound or compounds within and/or on the surface of the medical applicator known to those having skill in the art falls within the scope of the method disclosed herein.

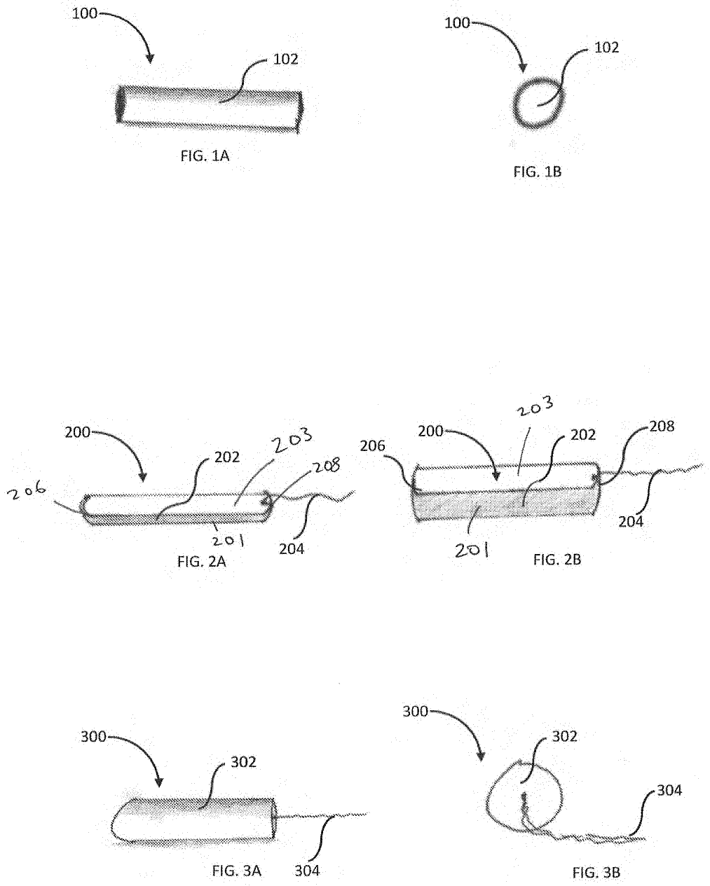

FIG. 1A is a side view of an embodiment of a medical applicator 100 that has undergone a method for reducing blood loss (e.g. a compound or pharmaceutical agent freeze dried on the outer surface or within the medical applicator). In one embodiment, the medical applicator 100 illustrated in FIG. 1A may be a nasal tampon. The medical applicator 100 may be elongate and include an outer surface 102 that is configured to make contact with a user's skin or wound to exert pressure on injured blood vessels. In some embodiments, the medical applicator 100 comprises an absorptive material, such as cotton, etc. The absorptive material is configured to absorb blood and expand as the absorptive material absorbs the blood. The expansion of the absorptive material is configured to increase the pressure on the wound when the medical applicator is disposed in the user's nasal cavity. In one embodiment, the medical applicator 100 may have an antifibrinolytic deposited on the outer surface 102 of the absorptive material of the medical applicator 100. The antifibrinolytic may cause injured blood vessels to reduce blood flow by promoting the blood to clot when the medical applicator 100 is used as intended (e.g., a nasal tampon placed within the nostril during a nosebleed). For example, the medical applicator 100 may be soaked in a solution of tranexamic avid and water which may deposit the tranexamic acid on the medical applicator 100 after an evaporation process, in one embodiment. The medical applicator 100 has about 250 mg of tranexamic acid.

In another embodiment, the medical applicator 100 may have a compound or pharmaceutical agent deposited on the outer surface 102 of the medical applicator 100 that causes vasoconstriction (e.g., Oxymetazoline). The compounds causing vasoconstriction may instigate the injured blood vessels to reduce blood flow by promoting the blood vessels to constrict when the medical applicator 100 is used as intended (e.g., a nasal tampon placed within the nostril during a nosebleed). In yet another embodiment, an antifibrinolytic compound and a compound causing vasoconstriction may be deposited onto the outer surface 102 of the medical applicator 100.

Various other compounds or pharmaceutical agents may also be used. For example, hyalauronic acid may be freeze dried on the absorptive material of the medical applicator 100. Hyalauronic acid may maintain water levels in the skin and mucosa in human tissue. Thus, the hyalauronic acid would thus prevent further drying and cracking of the friable nasal mucosa of the user.

It should be appreciated a plurality of compounds may be deposited on the outer surface 102 of the medical applicator 100. Compounds may also be deposited within the absorptive material of the medical applicator 100 in some embodiments. For example, the medical applicator 100 may be soaked in a solution of aminocaproic acid and water which may deposit an even concentration of the solution throughout the medical applicator 100.

FIG. 1B is a distal end view of an embodiment of a medical applicator 100 that has undergone a method for reducing blood loss (e.g. a compound or pharmaceutical agent freeze dried on the outer surface or within the medical applicator). It should be appreciated that the concentration, position, and process by which the compounds may be deposited onto and/or within the medical applicator 100 may vary. For example, at least one compound may be deposited on a distal end 102 of the medical applicator 100, in one embodiment. In another embodiment, at least one compound may be deposited on the distal end 102 and proximal end (not shown) of the medical applicator 100.

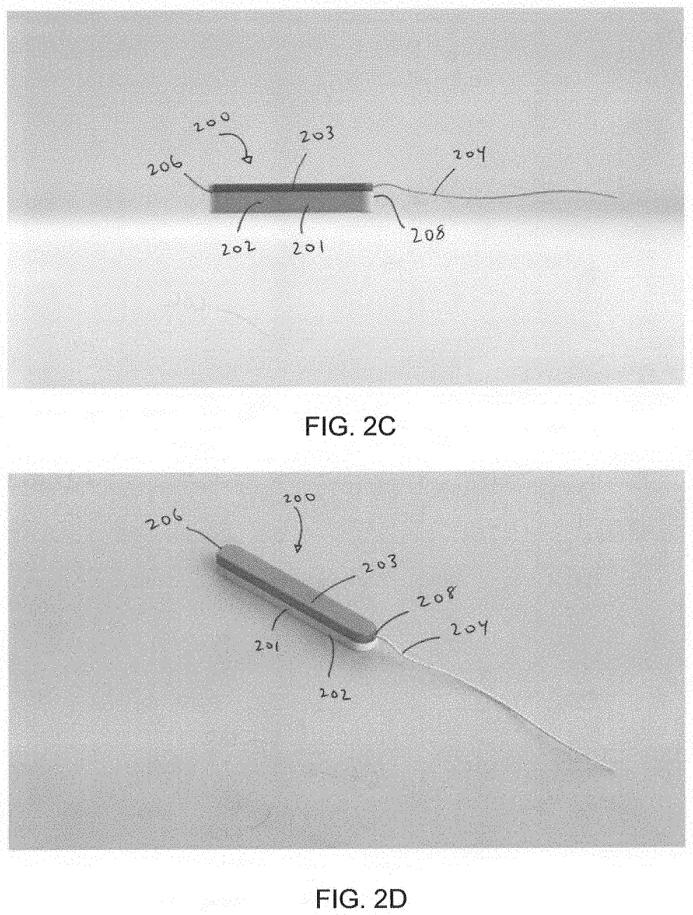

FIG. 2A is a side view of an embodiment of a medical applicator 200 that has undergone a method for reducing blood loss (e.g. a compound or pharmaceutical agent freeze dried on the outer surface or within the medical applicator). FIG. 2D is a perspective view of the medical applicator 200. In one embodiment, the medical applicator 200 illustrated in FIG. 2A may comprise an absorptive material 201, such as a dehydrated flat nasal insert, coupled to a rigid elongate member 203. The absorptive material 201 may extend from a proximal end 206 to a distal end 208 of the medical applicator 200. The absorptive material 201 may be a material that is configured to absorb blood from the user, such as cotton, etc. The rigid elongate member 203 may be rigid plastic or other similar material that provides a frame for the absorptive material 201. In one embodiment, the absorptive material 201 of the medical applicator 200 may include an outer surface 202 and a pull string 204 coupled to the proximal end 206 of the rigid elongate member 203. The pull string 204 may be used to remove the medical applicator 200 during or after use. The outer surface 202 of the absorptive material 201 is configured to make contact with a user's body at a wound site to exert pressure on injured blood vessels, such as within the nostril during a nosebleed. As the absorptive material 201 absorbs the blood, the absorptive material is configured to expand and thus exert additional pressure to the injured blood vessels.

In one embodiment, the medical applicator 200 may have an antifibrinolytic (e.g., aminocaproic acid) deposited on the outer surface 202 of the medical applicator 200. The antifibrinolytic may cause the injured blood vessels to reduce blood flow by promoting the blood to clot when the medical applicator 200 is used as intended (e.g., a flat nasal insert placed within the nostril during a nosebleed). In another embodiment, the medical applicator 200 may have a compound deposited on the outer surface 202 of the medical applicator 200 that causes vasoconstriction (e.g., Oxymetazoline). The compounds causing vasoconstriction may instigate the injured blood vessels to reduce blood flow by promoting the blood vessels to constrict when the medical applicator 200 is used as intended (e.g., a flat nasal insert placed within the nostril during a nosebleed). In yet another embodiment, an antifibrinolytic compound and a compound causing vasoconstriction may be deposited onto the outer surface 202 of the medical applicator 200. It should be appreciated that a plurality of compounds may be deposited on the outer surface 202 of the medical applicator 200. Compounds may also be deposited within the medical applicator 200 in some embodiments. For example, the medical applicator 200 may be soaked in a solution of tranexamic acid and water which may deposit an even concentration of the solution throughout the medical applicator 200 after an evaporation process, in one embodiment.

FIGS. 2B and 2C are side views of the medical applicator 200 that has undergone a method for reducing blood loss (e.g. a compound or pharmaceutical agent freeze dried on the outer surface or within the medical applicator). FIGS. 2B and 2C illustrate the absorptive material 201 of the medical applicator 200 in an expanded configuration after the absorptive material 201 has absorbed blood from the user. It should be appreciated that the concentration, position, and process by which the compounds may be deposited onto and/or within the medical applicator 200 may vary. For example, at least one compound may be deposited on a proximal end 206 of the medical applicator 200, in one embodiment. In another embodiment, at least one compound may be deposited on the distal end 208 and proximal end 206 of the medical applicator 200.

FIG. 3A is a side view of an embodiment of a medical applicator 300 that has undergone a method for reducing blood loss (e.g. a compound or pharmaceutical agent freeze dried on the outer surface or within the medical applicator). In one embodiment, the medical applicator 300 illustrated in FIG. 3A may be a vaginal tampon comprising an absorptive material. In one embodiment, the absorptive material of the medical applicator 300 may include an outer surface 302 and a pull string 304 coupled to a proximal end. The pull string 304 may be used to remove the medical applicator 300 during or after use. The outer surface 302 is configured to make contact with a user's body to absorb blood. In one embodiment, the medical applicator 300 may have an antifibrinolytic (e.g., tranexamic acid) deposited within the medical applicator 300 and also on the outer surface 302 of the medical applicator 300. The antifibrinolytic may reduce blood flow by promoting the blood to clot when the medical applicator 300 is used as intended. In another embodiment, the medical applicator 300 may have a compound deposited within the medical applicator 300 and on the outer surface 302 of the medical applicator 300 that causes vasoconstriction (e.g., Oxymetazoline). The compounds causing vasoconstriction may reduce blood flow by promoting the blood vessels to constrict when the medical applicator 300 is used as intended. In yet another embodiment, an antifibrinolytic compound and a compound causing vasoconstriction may be deposited within the medical applicator 300 and onto the outer surface 302 of the medical applicator 300. It should be appreciated a plurality of compounds may be deposited within the medical applicator 300 and on the outer surface 302 of the medical applicator 300.

FIG. 3B is a distal end view of an embodiment of a medical applicator 300 that has undergone a method for reducing blood loss (e.g. a compound or pharmaceutical agent freeze dried on the outer surface or within the medical applicator). It should be appreciated that the concentration, position, and process by which the compounds may be deposited onto and/or within the medical applicator 300 may vary. For example, at least one compound may be deposited on a distal end (not shown) of the medical applicator 300 and another compound may also be placed within the medical applicator 300 itself, in one embodiment.

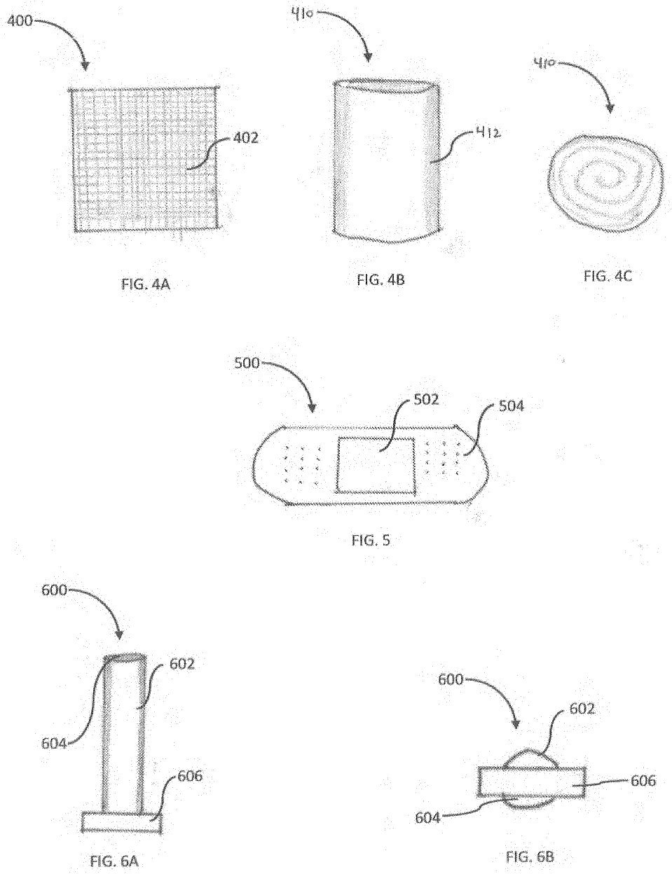

FIG. 4A is a side view of an embodiment of a medical applicator 400 that has undergone a method for reducing blood loss (e.g. a compound or pharmaceutical agent freeze dried on the outer surface or within the medical applicator). In one embodiment, the medical applicator 400 may be a piece of pre-cut gauze or another type of bandage wrap. In this embodiment, the compound that promotes reduced blood loss may deposit onto the surface 402 of the pre-cut medical applicator 400. Alternatively, the piece of material that comprises the medical applicator 400 may be cut from a larger roll of bandage that has undergone a method for reducing blood loss. FIG. 4B is a side view of an embodiment of a bandage roll 410 that has undergone a method for reducing blood loss (e.g. a compound or pharmaceutical agent freeze dried on the outer surface or within the medical applicator). FIG. 4C is end view of the bandage roll 410 that has undergone a method for reducing blood loss (e.g. a compound or pharmaceutical agent freeze dried on the outer surface or within the medical applicator).

FIG. 5 is a perspective view of an embodiment of a medical applicator 500 that has undergone a method for reducing blood loss (e.g. a compound or pharmaceutical agent freeze dried on the outer surface or within the medical applicator). In one embodiment, the medical applicator 500 depicted in FIG. 5 may be an adhesive bandage (e.g., a Band-Aid) having a pad 502 (e.g., a cotton pad) adhered to an adhesive strip 504. At least one compound to reduce blood loss may be deposited onto the pad 502 such that the at least one compound contacts the user's skin to cause blood vessels to reduce blood flow and/or the blood to clot when the medical applicator 500 is used for its intended purpose. It should be appreciated that the size and shape of the pad 502 and the size and shape of the adhesive strip 504 may vary. It should be further appreciated that the concentration of the compound deposited onto and/or within the pad 502 may also vary based on application. For example, there may be a rating system in which a lower rated bandage may have a lesser concentration of the compound than a higher rated bandage.

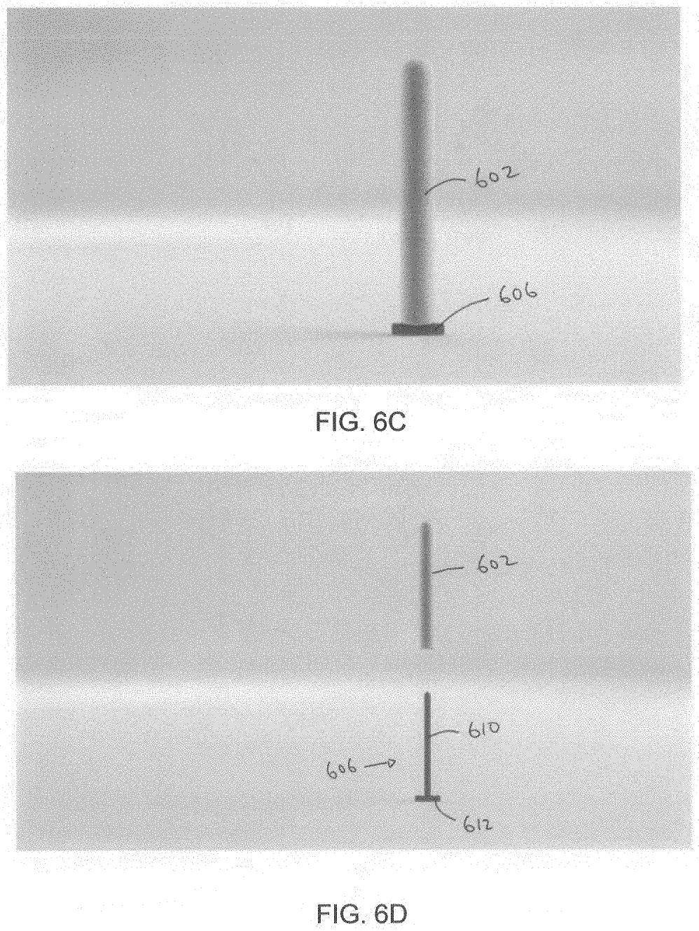

FIG. 6A is a side view of an embodiment of a medical applicator 600 that has undergone a method for reducing blood loss (e.g. a compound or pharmaceutical agent freeze dried on the outer surface or within the medical applicator). FIGS. 6B and 6C are bottom views of the medical applicator 600. In one embodiment, the medical applicator 600 depicted in FIG. 6A may be a nasal insert having an elongated member 602 having a distal end 604, and a stop member 606 disposed at a proximal end 608 of the elongated member 602. The stop member 606 may be a cross-bar that is orthogonal to the longitudinal axis of the elongated member 602 and may prevent the medical applicator 600 from inserting too deep within the user's nasal cavity. The length of the stop member 6060 is greater than the circumference of the elongated member 602 and greater than the nostril of the user or patient. Accordingly, the user may only insert the medical applicator 600 a predetermined distance before the stop member 606 engages with the nostril of the user and prevent further penetration of the medical applicator 600 into the user's nasal cavity. The elongated member 602 has a predetermined length that is configured to prevent the medical applicator 600 from being inserted too deep within the user's nasal cavity.

FIG. 6D illustrates an exploded view of the medical applicator 600 including the elongated member 602 and the stop member 606. The elongated member 602 comprises an absorptive material, such as cotton, for absorbing blood of the user. The stop member 606 comprises an elongate portion 610 and a cross bar 612. The cross-bar 612 is orthogonal to the elongate portion 610. The stop member 606 may be fabricated from a rigid material, such as plastic or other suitable material. The elongate portion 610 of the stop member 606 is disposed within the elongate member 602 and the cross-bar 612 is disposed outside of the elongate member 602.

The elongated member 602 may have an antifibrinolytic (e.g., aminocaproic acid) deposited on the outer surface of the elongated member 602. The antifibrinolytic may cause the injured blood vessels to reduce blood flow by promoting the blood to clot when the medical applicator 600 is used as intended (e.g., a nasal insert placed within the nostril during a nosebleed). In another embodiment, the medical applicator 600 may have a compound deposited on the outer surface of the elongated member 602 that causes vasoconstriction (e.g., Oxymetazoline). The compounds causing vasoconstriction may instigate the injured blood vessels to reduce blood flow by promoting the blood vessels to constrict when the medical applicator 600 is used as intended (e.g., a nasal insert placed within the nostril during a nosebleed). In yet another embodiment, an antifibrinolytic compound and a compound causing vasoconstriction may be deposited onto the outer surface of the elongated member 602. It should be appreciated a plurality of compounds may be deposited on the outer surface of the elongated member 602. Compounds may also be deposited within the elongated member 602 in some embodiments. For example, the elongated member 602 may be soaked in a solution of tranexamic acid and water which may deposit an even concentration of the solution throughout the elongated member 602 after an evaporation process, in one embodiment. It should be appreciated that the concentration, position, and process by which the compounds may be deposited onto and/or within the elongated member 602 may vary. For example, at least one compound may be deposited on a distal end 604 of the elongated member 602 and another compound may also be placed within the elongated member 602 itself, in one embodiment.

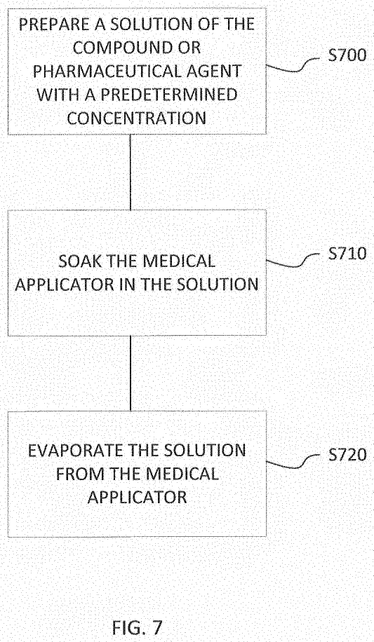

FIG. 7 illustrates a method of depositing compounds or pharmaceutical agents on an outer surface of within an absorptive material of a medical applicator. In S700, a solution containing the compound(s) or pharmaceutical agent(s) may be prepared. For example, the solution may be a solution of tranexamic acid with a concentration of 125 mg/mL.

In S710, a medical applicator may be soaked in the solution to have the absorptive material absorb the compound(s) or pharmaceutical agent(s). After the medical applicator is soaked, the medical applicator may be removed from the solution.

In S720, the liquid form the medical applicator is evaporated from the medical applicator. This may be accomplished via a lyophilizing (freeze-drying) process that removes the liquid but does not remove the compound(s) or pharmaceutical agent(s) from the medical applicator.

The claimed method may be embodied in other specific forms without departing from its spirit or essential characteristics. The described embodiments are to be considered in all respects only as illustrative and not restrictive. The scope of the claimed method is, therefore, indicated by the appended claims rather than by the foregoing description. All changes that come within the meaning and range of equivalency of the claims are to be embraced within their scope.

Many modifications and other embodiments of the invention set forth herein will come to mind to one skilled in the art to which this claimed method pertains and having the benefit of the teaching presented in the foregoing descriptions and the associated drawings. Therefore, it should be understood that the claimed method should not to be limited to the specific embodiments disclosed and that modifications and other embodiments are intended to be included within the scope of the appended claims. Although specific terms are employed herein, they are used in a generic and descriptive sense only, and not for purposes of limitation.

We claim:

1. An medical applicator comprising:
an absorptive material; and
a pharmaceutical agent that is freeze dried to the absorptive material of the medical applicator;
wherein the pharmaceutical agent is configured to prevent a user's body natural enzymatic or catalytic breakdown of clotted blood to bring about a physiological effect that reduces the user's blood loss.

2. The medical applicator of claim 1, wherein the pharmaceutical agent is deposited on an outer surface of the absorptive material.

3. The medical applicator of claim 1, wherein the pharmaceutical agent is deposited within the absorptive material of the medical applicator.

4. The medical applicator of claim 1, wherein the pharmaceutical agent is deposited within the absorptive material of the medical applicator and an outer surface of the absorptive material of the medical applicator.

5. The medical applicator of claim 1, wherein the pharmaceutical agent comprise an antifibrinolytic.

6. The medical applicator of claim 5, wherein the antifibrinolytic is tranexamic acid.

7. The medical applicator of claim 6, wherein the pharmaceutical agent comprises about 250 mg of tranexamic acid.

8. The medical applicator of claim 5, wherein the antifibrinolytic is aminocaproic acid.

9. The medical applicator of claim 1, wherein the pharmaceutical agent comprises a compound with vasoconstrictive characteristics.

10. The medical applicator of claim 9, wherein the compound with vasoconstrictive characteristics is oxymetazoline.

11. The medical applicator of claim 9, wherein the compound with vasoconstrictive characteristics is epinephrine.

12. The medical applicator of claim 1, wherein the medical applicator comprises a plurality of a pharmaceutical agents that are freeze dried to the absorptive material.

13. The medical applicator of claim 1, further comprising a cross-bar disposed at a proximal end of the absorptive material, wherein the cross-bar is configured to prevent the medical applicator from being lodged too deep in a nasal cavity.

14. The medical applicator of claim 13, further comprising a rigid elongate member and coupled to the cross-bar, wherein the absorptive material is elongate, and wherein the rigid elongate member is disposed within the absorptive material.

15. The medical applicator of claim 1, further comprising a pull string coupled to a proximal end of the medical applicator.

16. The medical applicator of claim 1, wherein the medical applicator further comprises a rigid elongate member that extends from a proximal end to a distal end of the medical applicator, wherein the absorptive material is coupled to the rigid elongate member and extends from the proximal end to the distal end of the applicator.

17. The medical applicator of claim 1, wherein the medical applicator is a vaginal tampon.

18. The medical applicator of claim 1, wherein the medical applicator is a bandage.

19. A method of treating a bloody nose comprising:
inserting a medical applicator into a nostril of a patient, wherein the medical applicator comprises:
an absorptive material; and
a pharmaceutical agent that is freeze dried to the absorptive material of the medical applicator;
wherein the pharmaceutical agent is configured to prevent the body's natural enzymatic or catalytic breakdown of clotted blood to bring about a physiological effect that reduces the user's blood loss.

20. A method of preparing a medical applicator with a freeze dried pharmaceutical agent comprising:
preparing a solution of a pharmaceutical agent with a predetermined concentration;
soaking a medical applicator in the solution, the medical applicator comprising an absorptive material; and
freeze drying the medical applicator to remove a liquid from the solution from the medical applicator but maintain the pharmaceutical agent in the absorptive material.
